(19) United States
(12) Patent Application Publication
(10) Pub. No.: US 2023/0001221 A1
(43) Pub. Date: Jan. 5, 2023

(54) Methods of Treating Cancer with Alternating Electric Fields, Checkpoint Inhibitors, and Combination Chemotherapy

(71) Applicant: Novocure GmbH, Root D4 (CH)

(72) Inventor: Ori FARBER, Haifa (IL)

(73) Assignee: Novocure GmbH, Root D4 (CH)

(21) Appl. No.: 17/852,007

(22) Filed: Jun. 28, 2022

Related U.S. Application Data
(60) Provisional application No. 63/216,864, filed on Jun. 30, 2021.

(51) Int. Cl. A61N 1/40; A61K 39/395; A61K 31/7068; A61K 31/337; A61P 35/00

(52) U.S. Cl. CPC A61N 1/40; A61K 39/3955; A61K 31/7068; A61K 31/337; A61P 35/00

(57) ABSTRACT

Methods of treating cancer are provided. In some instances, the method comprises applying alternating electric fields to the abdomen of the subject at a frequency of 100 to 500 kHz, administering a checkpoint inhibitor to the subject, and administering systemic cancer therapy to the subject. In some instances, the cancer is pancreatic ductal adenocarcinoma.

### CROSS REFERENCE TO RELATED APPLICATIONS

This application claims the benefit of U.S. Provisional Application 63/216,864, filed Jun. 30, 2021, which is incorporated herein by reference in its entirety.

### BACKGROUND

Pancreatic ductal adenocarcinoma (PDAC) is typically diagnosed late, when curative resection is not feasible and prognosis is grim, with less than 5% of patients surviving 5 years^{1, 2}. During the past decades, two combinations of chemotherapy—FOLFIRINOX (oxaliplatin, folinic acid, irinotecan and fluorouracil) and albumin-bound paclitaxel (nab-paclitaxel) with gemcitabine have gained acceptance as front-line treatments (e.g., standard of care)^{1, 3-5}. However, response rates are not high, duration of response is short and progression after first line is inevitable.

Immunotherapy has revolutionized cancer care in many types of cancer in recent years. Immune-checkpoint inhibitors targeting CTLA-4, PD-1 and PD-L1 have the potential to reverse the anti-inflammatory tumor microenvironment and aid the immune system elicit an anti-tumoral response. FDA approved immune-checkpoint inhibitors, as ipilimumab, nivolumab, pembrolizumab and atezolizumab have shown to prolong survival in many cancer patients. However, immunotherapy has failed to improve the outcome of patients in some cancer types, notably PDAC⁶.

Tumor treating fields (TTFields) are a non-invasive, regional antimitotic treatment modality with minimal toxicity which have been approved for the treatment of recurrent and newly diagnosed glioblastoma (GBM) and malignant pleural mesothelioma (MPM) by the Food and Drug Administration (FDA) and have obtained a CE mark for marketing in Europe for the same indications. TTFields act by delivering low intensity (e.g., 1-3 V/cm), intermediate frequency (e.g., 100-300 kHz), alternating electric fields to the tumor using non-invasive transducer arrays placed on the skin around the region of the body containing the tumor. TTFields act predominantly during two phases of mitosis: 1) during metaphase, by disrupting the formation of the mitotic spindle, and 2) during cytokinesis, by dielectrophoretic dislocation of intracellular constituents resulting in apoptosis. Accumulating evidence demonstrate additional anti-neoplastic mechanisms for TTFields, such as interference with DNA repair mechanism, autophagy and migratory properties, as well as increasing the permeability of cells. Importantly, TTFields were shown to enhance antitumor immunity by stimulating macrophages to secrete reactive oxygen species and proinflammatory cytokines, promoting dendritic cell recruitment and maturation, resulting in accumulation of CD4+ and CD8+ at the tumor site.⁷⁻¹⁴.

### SUMMARY

Aspects described herein provide a method of treating cancer in a subject diagnosed with or suspected of having cancer by applying alternating electric fields to the abdomen of the subject at a frequency of 50 kHz to 10 MHz, administering a checkpoint inhibitor to the subject, and administering systemic cancer therapy to the subject. In some instances, at least a portion of the applying step is performed simultaneously with at least a portion of the delivering step.

### BRIEF DESCRIPTION OF THE DRAWINGS

FIG. 1 shows an exemplary study scheme for a method of treating a subject having metastatic cancer.

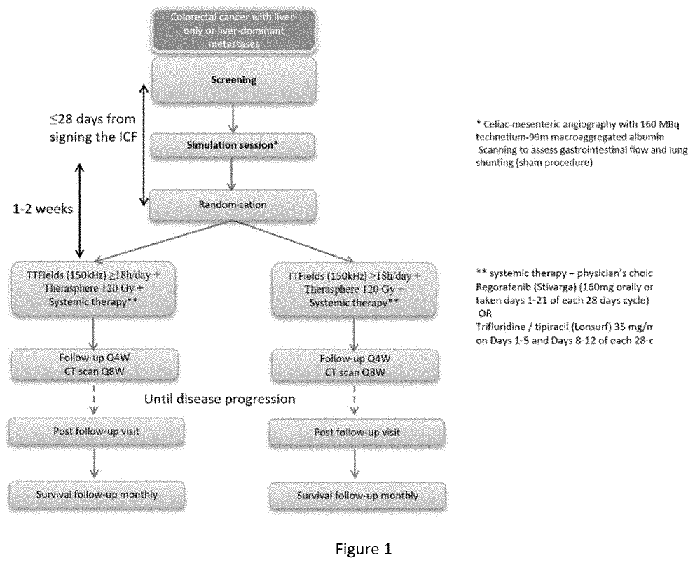

### DESCRIPTION

TTFields were shown to inhibit proliferation of human and murine pancreatic cancer cell lines¹⁵. In-vitro result have also shown enhanced cytotoxicity when combined with chemotherapies such as gemcitabine, irinotecan, 5-FU and paclitaxel. Moreover, in-vivo experiments have shown that adding TTFields to chemotherapy (5-FU or gemcitabine) resulted in a significant inhibition of tumor growth¹⁵.

Based on these results, the PANOVA study (NCT01971281) was the first study testing TTFields in pancreatic patients16. Forty patients with advanced pancreatic cancer were enrolled to receive TTFields with gemcitabine (n=20) or TTFields with nab-paclitaxel plus gemcitabine (n=20).

No increase in serious AEs (SAES) was observed compared to that anticipated with systemic chemotherapy alone and no TTFields-related SAEs were reported16. In patients receiving TTFields with nab-paclitaxel plus gemcitabine, the median PFS was 12.7 months (95% CI 5.4, NA). Median PFS was not reached in the locally advanced disease group and was 9.3 months in metastatic disease.

Taken together, adding TTFields and atezolizumab to standard gemcitabine/nab-paclitaxel chemotherapy, is hypothesized to increase disease control rate, and ultimately prolonging survival in this patient population.

Aspects described herein provide a method of treating cancer in a subject diagnosed with or suspected of having cancer by applying alternating electric fields to the abdomen of the subject at a frequency of 50 kHz to 10 MHz, administering a checkpoint inhibitor to the subject, and administering systemic cancer therapy to the subject. In some instances, at least a portion of the applying step is performed simultaneously with at least a portion of the delivering step.

In some aspects, the cancer is selected from the group consisting of pancreatic ductal adenocarcinoma, pancreatic cancer, liver cancer, breast cancer, ovarian cancer, cervical cancer, endometrial carcinoma, colorectal cancer, bladder cancer, biliary cancer, renal cell carcinoma, gastric cancer, esophageal cancer, urothelial carcinoma, and melanoma. In some aspects, the cancer is pancreatic ductal adenocarcinoma. In another aspect, the cancer is an abdominal cancer.

In some aspects, the frequency of the alternating electric fields is from 50 kHz to 10 MHz, 50 kHz to 1 MHz, 100 kHz to 500 kHz, 120 kHz to 180 kHz. In some aspects, the frequency of the alternating electric fields is 150 kHz.

In some aspects, the intensity of the alternating electric fields is 0.1 V/cm to 20 V/cm. In some aspects, the intensity is 1.0 V/cm to 4 V/cm.

In some aspects, the checkpoint inhibitor is selected from the group consisting of PD-1 inhibitors, PD-L1 inhibitors, CTLA-4 inhibitors.

In some aspects, the checkpoint inhibitor is selected from the group consisting of atezolizumab, pembrolizumab, nivolumab, durvalumab, ipilimumab, dostarlimab, avelumab, tremelimumab, and cemiplimab. In some instances, the checkpoint inhibitor is atezolizumab.

In some aspects, the atezolizumab is administered every four weeks. In some instances, the dose of the atezolizumab is 1680 mg administered every four weeks. In some instances, the dose of the checkpoint inhibitor can be at least 10, at least 20, at least 30, at least 40, at least 50, at least 60, at least 70, at least 80, or at least 90 percent lower than the standard of care dose when used in combination with alternating electric fields and systemic therapy as described herein.

In some aspects, the systemic cancer therapy comprises one or more of gemcitabine and nab-paclitaxel. In some instances, the systemic cancer therapy comprises gemcitabine. In some instances, the systemic cancer therapy comprises nab-paclitaxel. In some instances, the systemic cancer therapy comprises gemcitabine and nab-paclitaxel.

In some aspects, the nab-paclitaxel is administered on days 1, 8, and 15 of each 28 day cycle. In some instances, the gemcitabine is administered on days 1, 8, and 15 of each 28 day cycle.

In some aspects, the dose of gemcitabine is 100 mg/m2 on days 1, 8, and 15 of each 28 day cycle. In some instances, the dose of nab-paclitaxel is 125 mg/m2 on days 1, 8, and 15 of each 28 day cycle. In some instances, the dose of the systemic therapy (e.g., gemcitabine, nab-paclitaxel) can be at least 10, at least 20, at least 30, at least 40, at least 50, at least 60, at least 70, at least 80, or at least 90 percent lower than the standard of care dose when used in combination with alternating electric fields and systemic therapy as described herein.

In some aspects, the alternating electric fields are applied to the abdomen or torso of the subject.

In some aspects, the alternating electric fields are applied to the abdomen or torso of the subject for at least 3 hours. In some instances, the alternating electric fields are applied to the abdomen or torso of the subject for at least 18 hours.

In some aspects, the alternating electric fields are applied prior to or during the administering of the checkpoint inhibitor. In some instances, the alternating electric fields are applied prior to or during the administering of the systemic cancer therapy.

The term “administering systemic cancer therapy” refers to providing the systemic cancer therapy (e.g., chemotherapeutic agent) to a patient by a healthcare professional or the patient through any suitable and accepted route of administration (e.g., oral, intravenous, parenteral, topical etc.) as approved on the product label by a regulatory authority, or as part of an approved clinical trial. Prescribing a checkpoint inhibitor can also be “administering” a checkpoint inhibitor.

Alternating electric fields can be applied continuously or discontinuously. The term “continuously” refers to applying alternating electric fields for a substantially constant period of time. Continuous application of alternating electric fields can occur even if the application is discontinued for a short period of time (e.g., seconds) in order to position equipment appropriately, or if there is a brief disruption of power.

The term “discontinuously” refers to applying alternating electric fields for a period of time with a periodic break or disruption for seconds, minutes, an hour, days or more. In this aspect, a patient could apply alternating electric fields for a period of time (e.g., 1, 2, 3, 4, 8, 24, 48, or 72 hours) with a 15 minute, 30 minute, 45 minute, or 1 hour period without applying the alternating electric field. In another aspect, the patient could apply the alternating field continuously while sleeping and discontinuously while awake. In a further aspect, the patient can apply the alternating electric field continuously except during mealtime or during a social event.

In some aspects, alternating electric fields are applied to an organ located in an abdomen or torso of the subject (e.g., liver, pancreas, bile duct, and spleen). In some instances of the first method, the alternating electric fields are applied after or during the administering of the systemic cancer therapy.

In some aspects of the first method, the chemotherapeutic agent or agents are determined to be the “standard of care” for a particular type of primary or metastatic cancer. In some instances of the first method, the chemotherapeutic agent or agents are determined to be the “standard of care” for pancreatic cancer.

The non-limiting example below illustrates how to make and use aspects described herein and provide additional supporting data, with reference to the FIGURE, for the embodiments and aspects described herein, including modifications and alternations. Without being bound by any theories or hypotheses, the examples may include possible explanations for the described data. Accordingly, it is intended that the present invention not be limited to the examples provided below, but that it has the full scope defined by the language of the claims listed below, and equivalents thereof.

### Example

An exemplary multicenter, single arm, open-label study of Tumor Treating Fields (TTFields) at 150 kHz to the abdomen is conducted using the NovoTTF-200T System concomitant with IV atezolizumab, nab-paclitaxel and gemcitabine in subjects previously untreated for their PDAC. Approximately 76 subjects are enrolled in this study for examination of the effectiveness and safety of TTFields concomitant with atezolizumab, nab-paclitaxel and gemcitabine. Subjects are enrolled to receive TTFields at 150 kHz to the abdomen using the NovoTTF-200T System for at least 18 hours a day on average concomitant with atezolizumab 1680 mg IV infusion Q4W, nab-paclitaxel 125 mg/m² IV infusion on days 1, 8, and 15 of each 28 day cycle and gemcitabine 1000 mg/m² IV infusion on days 1, 8, and 15 of each 28 day cycle.

Subjects are evaluated every 4 weeks (28±7 days) clinically and every 8 weeks (56±7 days) with radiographic imaging to assess response to treatment. All imaging obtained on study are assessed by the site using Response Evaluation Criteria in Solid Tumors (RECIST) v1.1 for determination of DCR, ORR, PFS, PFS6, and DOR. Adverse event (AE) monitoring is ongoing throughout the study and graded in severity according to the guidelines outlined in the National Cancer Institute (NCI) Common Terminology Criteria for Adverse Events (CTCAE) version 5.0. Treatment with TTFields, atezolizumab, nab-paclitaxel and gemcitabine continues until documented disease progression, unacceptable adverse event(s), intercurrent illness that prevents further administration of treatment, investigator's decision to withdraw the subject, subject withdraws consent, pregnancy of the subject, noncompliance with study treatment or procedure requirements, or administrative reasons.

After the end of treatment, each subject is followed for a minimum of 30 days for AE monitoring. Serious adverse events (SAES) are collected for up to 90 days following cessation of treatment or until the subject initiates new anticancer therapy, whichever is earlier. Subjects have post-treatment follow-up for disease status, including initiating a non-study anti-cancer treatment and experiencing disease progression, until death, withdrawing consent, or becoming lost to follow-up.

The primary endpoint of the study is DCR at 16 weeks by RECIST 1.1. Secondary endpoints include PFS, PFS6, 1-year survival rate, DOR and safety. Exploratory analyses include relationship between study treatments and biomarkers predicting response and tumor characteristics before and after treatment.

PD-L1 expression status and microsatellite instability (MSI) high status are evaluated on available samples.

A pilot, single arm, open-label study of Tumor Treating Fields (TTFields, 150 kHz) concomitant with atezolizumab, gemcitabine and nab-paclitaxel for first-line (1 L) treatment of metastatic pancreatic ductal adenocarcinoma (PDAC) are conducted on patients having previously untreated metastatic PDAC (mPDAC).

Primary Objective:

To evaluate the disease control rate (DCR) at 16 weeks by RECIST 1.1 in subjects with 1 L mPDAC treated with TTFields concomitant with atezolizumab, gemcitabine and nab-paclitaxel.

Secondary Objectives:

(1) To evaluate the overall survival (OS) in subjects with 1 L mPDAC treated with TTFields concomitant with atezolizumab, gemcitabine and nab-paclitaxel.

(2) To evaluate the progression free survival (PFS) by RECIST v1.1 in subjects with 1 L mPDAC treated with TTFields concomitant with atezolizumab, gemcitabine and nab-paclitaxel.

(3) To evaluate the 1-year survival rate in subjects with 1 L mPDAC treated with TTFields concomitant with atezolizumab, gemcitabine and nab-paclitaxel.

(4) To evaluate the objective response rate (ORR) by RECIST v1.1 in subjects with 1 L mPDAC treated with TTFields concomitant with atezolizumab, gemcitabine and nab-paclitaxel.

(5) To evaluate the PFS rate at 6 months (PFS6) in subjects with 1 L mPDAC treated with TTFields concomitant with atezolizumab, gemcitabine and nab-paclitaxel.

(6) To evaluate the duration of response (DOR) by RECIST v1.1 in subjects with 1 L mPDAC treated with TTFields concomitant with atezolizumab, gemcitabine and nab-paclitaxel.

(7) To evaluate the safety and tolerability profile of TTFields concomitant with atezolizumab, gemcitabine and nab-paclitaxel in subjects with 1 L mPDAC.

Exemplary Treatment Regimen

TTFields at 150 kHz to the abdomen, continuous for at least 18 hours a day on average, using the NovoTTF-200T System.

AND

Atezolizumab 1680 mg IV every 4 weeks (Q4W).

AND

Nab-paclitaxel 125 mg/m2 IV infusion on Days 1, 8, and 15 of each 28 day cycle

AND

Gemcitabine 1000 mg/m2 IV infusion on Days 1, 8, and 15 of each 28 day cycle

Disease control rate (DCR) is evaluated at 16 weeks following the administration of gemcitabine and nab-paclitaxel, and was 48% in the phase 3 historical control study³. In order to detect DCR of 63% in NovoTTF-200T concomitant with atezolizumab, gemcitabine and nab-paclitaxel treated subjects compared to the DCR of 48% historical control, a sample size of 76 subjects is required to achieve a power of 80% at a one-sided alpha level of 0.05 using one sample exact test for proportion, considering a loss to follow-up of up to 10%. The sample size was calculated in NCSS/PASS11 16.04 software.

The anticipated time of accrual is 24 months, and the total time of the study is 36 months. Expected 12 months on the study.

Exemplary Inclusion Criteria:
18≥years
Eastern Cooperative Oncology Group (ECOG) performance status of 0 or 1
Histologically or cytologically confirmed de novo diagnosis of metastatic pancreatic ductal adenocarcinoma
No prior treatment for PDAC
Life expectancy greater than or equal to 3 months
Measurable disease, as defined by RECIST v1.1
Amenable and assigned by the investigator to receive therapy with gemcitabine and nab-paclitaxel
Able to operate the NovoTTF200T system independently or with the help of a caregiver.
Signed informed consent form for the study protocol

Exemplary exclusion criteria:
Known CNS metastases or leptomeningeal disease
Prior palliative treatment (e.g., surgery, radiation) to the tumor
Uncontrolled pleural effusion, pericardial effusion, or ascites requiring drainage procedure (i.e., more than one time per month)
Active or history of autoimmune disease or immune deficiency
History of idiopathic pulmonary fibrosis, organizing pneumonia, drug-induced pneumonitis, or idiopathic pneumonitis, or evidence of active pneumonitis on screening chest computed tomography (CT) scan
Positive human immunodeficiency (HIV) test at screening or at any time prior to screening
Active hepatitis B or C virus infection or active tuberculosis
Severe infection within 4 weeks prior to initiation of study treatment
Prior allogeneic stem cell or solid organ transplantation
Prior malignancies treated primarily or for recurrence within 2 years prior to inclusion in this study, except for completely resected non-melanomatous skin carcinoma or successfully treated in situ carcinoma of the skin or cervix of the uterus
Clinically significant (as determined by the investigator) hematological, hepatic and renal dysfunction, defined as: Neutrophil count<1.5×10\hat{ }9/L and platelet count<100×10\hat{ }9/L; bilirubin>1.5× Upper Limit of Normal (ULN); AST and/or ALT>2.5×ULN; Patients with documented liver metastases: AST and/or ALT>5×ULN; and serum creatinine>1.5×ULN.
History of significant cardiovascular disease unless the disease is well controlled. Significant cardiac disease includes second/third degree heart block; significant ischemic heart disease; poorly controlled hypertension; congestive heart failure of the New York Heart Association (NYHA) Class II or worse (slight limitation of physical activity; comfortable at rest, but ordinary activity results in fatigue, palpitation or dyspnea).
History of arrhythmia that is symptomatic or requires treatment. Patients with atrial fibrillation or flutter controlled by medication are not excluded from participation in the study.
History of cerebrovascular accident (CVA) within 6 months prior to randomization or that is not stable.
Serious underlying medical condition that would impair the ability of the subject to receive protocol therapy.
History of any psychiatric condition that might impair patient's ability to understand or comply with the requirements of the study or to provide consent.
Concurrent anti-tumor therapy beyond gemcitabine and nab-paclitaxel
Implantable electronic medical devices in the torso, such as pacemakers
Known allergies to medical adhesives or hydrogel, or to one of the chemotherapies used in this study.
Pregnancy or breast-feeding (female patients with reproductive potential and their partners must accept to use effective contraception throughout the entire study period and for 3 months after the end of treatment). All patients who are capable of becoming pregnant must take a pregnancy test which is negative within 72 hours before beginning treatment. The definition of effective contraception is left up to the decision of the investigator.
Unable to follow the protocol for medical, psychological, familial, geographic or other reasons.

### REFERENCES

1. Wang-Gillam A, Li C P, Bodoky G, et al. Nanoliposomal irinotecan with fluorouracil and folinic acid in metastatic pancreatic cancer after previous gemcitabine-based therapy (NAPOLI-1): a global, randomised, open-label, phase 3 trial. Lancet. Feb. 6, 2016; 387(10018):545-557. doi:10.1016/s0140-6736(15)00986-1
2. Tempero M A. NCCN clinical practice guidelines in oncology (NCCN guidelines), Pancreatic adenocarcinoma. 2021; version 1.2021
3. Von Hoff D D, Ervin T, Arena F P, et al. Increased Survival in Pancreatic Cancer with nab-Paclitaxel plus Gemcitabine. New England Journal of Medicine. Oct. 31 2013; 369(18):1691-1703. doi:10.1056/NEJMoa1304369
4. Conroy T, Desseigne F, Ychou M, et al. FOLFIRINOX versus Gemcitabine for Metastatic Pancreatic Cancer. New England Journal of Medicine. May 12, 2011; 364(19):1817-1825. doi:10.1056/NEJMoa1011923
5. Ducreux M, Cuhna A S, Caramella C, et al. Cancer of the pancreas: ESMO Clinical Practice Guidelines for diagnosis, treatment and follow-up<sup>†</sup>. Annals of Oncology. 2015; 26:v56-v68. doi:10.1093/annonc/mdv295
6. Hilmi M, Bartholin L, Neuzillet C. Immune therapies in pancreatic ductal adenocarcinoma: Where are we now? World J Gastroenterol. May 28, 2018; 24(20):2137-2151. doi:10.3748/wjg.v24.i20.2137
7. Giladi M, Schneiderman R S, Voloshin T, et al. Mitotic Spindle Disruption by Alternating Electric Fields Leads to Improper Chromosome Segregation and Mitotic Catastrophe in Cancer Cells. Scientific Reports. Dec. 11, 2015; 5(1):18046. doi:10.1038/srep18046
8. Kirson E D, Gurvich Z, Schneiderman R, et al. Disruption of cancer cell replication by alternating electric fields. Cancer Res. May 1, 2004; 64(9):3288-95. doi:10.1158/0008-5472.can-04-0083
9. Kirson E D, Dbalý V, Tovaryš F, et al. Alternating electric fields arrest cell proliferation in animal tumor models and human brain tumors. Proceedings of the National Academy of Sciences. 2007; 104(24):10152. doi:10.1073/pnas.0702916104
10. Rominiyi O, Vanderlinden A, Clenton S J, Bridgewater C, Al-Tamimi Y, Collis S J. Tumour treating fields therapy for glioblastoma: current advances and future directions. British Journal of Cancer. Nov. 4, 2020; doi:10.1038/s41416-20-01136-5
11. Giladi M, Voloshin T, Shteingauz A, et al. Alternating electric fields (TTFields) induce immunogenic cell death resulting in enhanced antitumor efficacy when combined with anti-PD-1 therapy. The Journal of Immunology. 2016; 196(1 Supplement):75.26.
12. Silginer M, Weller M, Stupp R, Roth P. Biological activity of tumor-treating fields in preclinical glioma models. Cell Death & Disease. Apr. 1, 2017; 8(4):e2753-e2753. doi:10.1038/cddis.2017.171
13. Park J-I, Song K-H, Jung S-Y, et al. Tumor-Treating Fields Induce RAW264.7 Macrophage Activation Via NK-κB/MAPK Signaling Pathways. Technology in Cancer Research & Treatment. Jan. 1, 2019; 18:1533033819868225. doi:10.1177/1533033819868225
14. Kirson E D, Giladi M, Gurvich Z, et al. Alternating electric fields (TTFields) inhibit metastatic spread of solid tumors to the lungs. Clin Exp Metastasis. 2009; 26(7):633-40. doi:10.1007/s10585-009-9262-y
15. Giladi M, Schneiderman R S, Porat Y, et al. Mitotic disruption and reduced clonogenicity of pancreatic cancer cells in vitro and in vivo by tumor treating fields. Pancreatology. Jan. 1, 2014; 14(1):54-63. doi: https://doi.org/10.1016/j.pan.2013.11.009
16. Rivera F, Benavides M, Gallego J, Guillen-Ponce C, Lopez-Martin J, Kung M. Tumor treating fields in combination with gemcitabine or gemcitabine plus nab-paclitaxel in pancreatic cancer: Results of the PANOVA phase 2 study. Pancreatology. Jan. 1, 2019; 19(1):64-72. doi: https://doi.org/10.1016/j.pan.2018.10.004

All references cited herein, including but not limited to patents and patent applications, are incorporated herein by reference in their entirety.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

1. A method of treating cancer in a subject diagnosed with or suspected of having cancer comprising:
applying alternating electric fields to an abdomen or torso of the subject at a frequency of 50 kHz to 10 MHz;
administering a checkpoint inhibitor to the subject; and
administering systemic cancer therapy to the subject.

2. The method claim 1, wherein the cancer is selected from the group consisting of pancreatic ductal adenocarcinoma, pancreatic cancer, liver cancer, breast cancer, ovarian cancer, cervical cancer, endometrial carcinoma, colorectal cancer, bladder cancer, biliary cancer, renal cell carcinoma, gastric cancer, esophageal cancer, urothelial carcinoma, and melanoma.

3. The method of claim 2, wherein the cancer is pancreatic cancer.

4. The method of claim 2, wherein the cancer is pancreatic ductal adenocarcinoma.

5. The method of claim 1, wherein the frequency of the alternating electric fields is from 100 kHz and 500 kHz.

6. The method of claim 5, wherein the frequency of the alternating electric fields is from 120 kHz to 180 kHz.

7. The method of claim 1, wherein the frequency of the alternating electric fields is 150 kHz.

8. The method of claim 1, wherein the intensity of the alternating electric fields is 0.1 V/cm to 20 V/cm.

9. The method of claim 8, wherein the intensity is 1.0 V/cm to 4 V/cm.

10. The method of claim 1, wherein the checkpoint inhibitor is selected from the group consisting of PD-1 inhibitors, PD-L1 inhibitors, and CTLA-4 inhibitors.

11. The method of claim 1, wherein the checkpoint inhibitor is selected from the group consisting of atezolizumab, pembrolizumab, nivolumab, durvalumab, ipilimumab, dostarlimab, avelumab, tremelimumab, and cemiplimab.

12. The method of claim 11, wherein the checkpoint inhibitor is atezolizumab.

13. The method of claim 12, wherein the atezolizumab is administered every four weeks.

14. The method of claim 13, wherein a dose of the atezolizumab is 1680 mg administered every four weeks.

15. The method of claim 1, wherein the systemic cancer therapy comprises one or more of gemcitabine and nab-paclitaxel.

16. The method of claim 1, wherein the systemic cancer therapy comprises gemcitabine and nab-paclitaxel.

17. The method of claim 15, wherein nab-paclitaxel is administered on days 1, 8, and 15 of each 28 day cycle.

18. The method of claim 15, wherein the gemcitabine is administered on days 1, 8, and 15 of each 28 day cycle.

19. The method of claim 18, wherein dose of gemcitabine is 100 mg/m² on days 1, 8, and 15 of each 28 day cycle.

20. The method of claim 16, wherein dose of nab-paclitaxel is 125 mg/m² on days 1, 8, and 15 of each 28 day cycle.

21. The method of claim 1, wherein the alternating electric fields are applied to a torso of the subject.

22. The method of claim 1, wherein the alternating electric fields are applied to an abdomen of the subject for at least 3 hours.

23. The method of claim 22, wherein the alternating electric fields are applied to an abdomen of the subject for at least 18 hours.
